(19) United States
(12) Patent Application Publication
(10) Pub. No.: US 2026/0055101 A1
(43) Pub. Date: Feb. 26, 2026

(54) PREPARATION OF P2X3 ANTAGONIST

(71) Applicant: GlaxoSmithKline Intellectual Property (No.3) Limited, Stevenage (GB)

(72) Inventors: Karine VILLENEUVE, Stevenage (GB); David R. KRONENTHAL, Stevenage (GB)

(73) Assignee: GlaxoSmithKline Intellectual Property (No.3) Limited, Stevenage (GB)

(21) Appl. No.: 19/104,399

(22) PCT Filed: Aug. 23, 2023
(86) PCT No.: PCT/IB2023/058380

§ 371 Date: Feb. 18, 2025

Related U.S. Application Data
(60) Provisional application No. 63/373,532, filed on Aug. 25, 2022.

(51) Int. Cl. C07D 471/04; A61K 31/5377; C07D 265/30

(52) U.S. Cl. CPC C07D 471/04; A61K 31/5377; C07D 265/30

(57) ABSTRACT

Described herein are processes for the synthesis of P2X3 antagonists, wherein the P2X3 antagonist is methyl (S)-2-((2-(2,6-difluoro-4-(methylcarbamoyl)phenyl)-7-methylimidazo[1,2-a]pyridin-3-yl)methyl)morpholine-4-carboxylate (Compound 1), or a pharmaceutically acceptable salt or co-crystal thereof.

### BACKGROUND OF THE INVENTION

P2X purinoreceptors are a family of ion channels that are activated by extracellular adenosine triphosphate (ATP). Purinoreceptors have been implicated in a variety of biological functions. The P2X3 receptor subunit is a member of this family. It was originally cloned from rat dorsal root ganglia. Chen et al., Nature, vol. 377, pp. 428-431 (1995). The nucleotide and amino acid sequences of both rat and human P2X3 are now known. Lewis, et al., Nature, vol. 377, pp. 432-435 (1995); and Garcia-Guzman, et al., Brain Res. Mol. Brain Res., vol. 47, pp. 59-66 (1997).

### SUMMARY OF THE INVENTION

Described herein are processes for the synthesis of P2X3 antagonists, wherein the P2X3 antagonist is methyl (S)-2-((2-(2,6-difluoro-4-(methylcarbamoyl)phenyl)-7-methylimidazo[1,2-a]pyridin-3-yl)methyl)morpholine-4-carboxylate (Compound 1), or a pharmaceutically acceptable salt or co-crystal thereof. Various intermediates mentioned herein can be prepared according to methods disclosed in WO 2021/161109 and WO 2023/021328.

In one aspect described herein is a process for the preparation of methyl (S)-2-((2-(2,6-difluoro-4-(methylcarbamoyl)phenyl)-7-methylimidazo[1,2-a]pyridin-3-yl)methyl)morpholine-4-carboxylate (Compound 1), comprising:
A) the reaction of a compound with the structure:

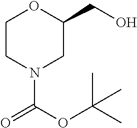

with 2,2,6,6-tetramethylpiperidine 1-oxyl or T3P to produce a compound with the structure:

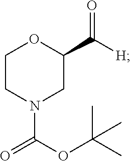

B) followed by the reaction of the compounds with the structures:

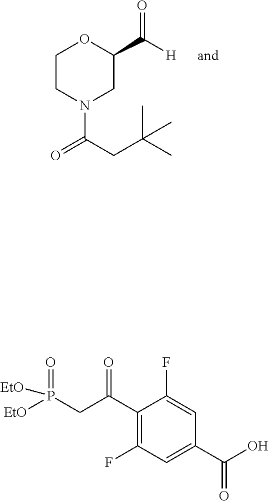 [SMILES: CC(C)(C)CC(=O)N1CCO[C@@H](C=O)C1 ; CCOP(=O)(CC(=O)c1c(F)cc(C(=O)O)cc1F)OCC]

with a mixture of potassium carbonate and potassium bicarbonate or potassium phosphate to produce a compound with the structure:

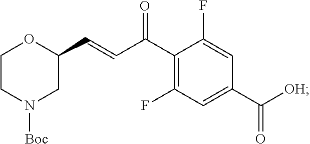 [SMILES: CC(C)(C)OC(=O)N1CCO[C@@H](/C=C/C(=O)c2c(F)cc(C(=O)O)cc2F)C1]

C) followed by the reaction of the compound with the structure:

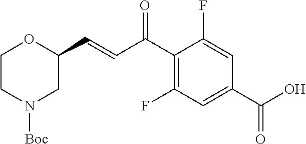 [SMILES: CC(C)(C)OC(=O)N1CCO[C@@H](/C=C/C(=O)c2c(F)cc(C(=O)O)cc2F)C1]

with palladium on carbon and hydrogen to produce a compound with the structure:

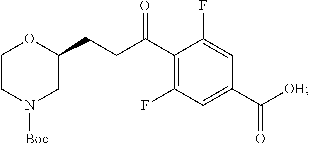 [SMILES: CC(C)(C)OC(=O)N1CCO[C@@H](CCC(=O)c2c(F)cc(C(=O)O)cc2F)C1]

D) followed by the reaction of the compound with the structure:

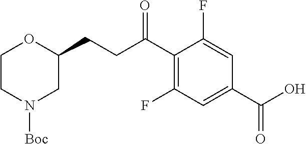 [SMILES: CC(C)(C)OC(=O)N1CCO[C@@H](CCC(=O)c2c(F)cc(C(=O)O)cc2F)C1]

with hydrogen chloride in ethyl acetate to produce a compound with the structure:

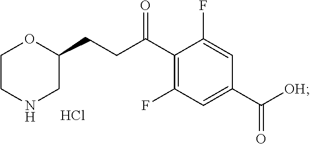 [SMILES: O=C(O)c1cc(F)c(C(=O)CC[C@H]2CNCCO2)c(F)c1 ; Cl]

E) followed by the reaction of the compound with the structure:

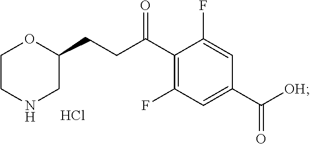 [SMILES: O=C(O)c1cc(F)c(C(=O)CC[C@H]2CNCCO2)c(F)c1 ; Cl]

with methyl chloroformate and aqueous sodium bicarbonate to produce a compound with the structure:

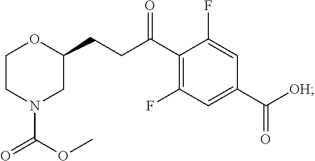 [SMILES: COC(=O)N1CCO[C@@H](CCC(=O)c2c(F)cc(C(=O)O)cc2F)C1]

F) followed b the reaction of the compound with the structure:

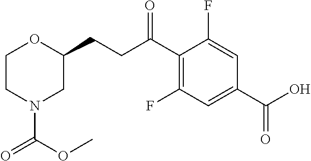 [SMILES: COC(=O)N1CCO[C@@H](CCC(=O)c2c(F)cc(C(=O)O)cc2F)C1]

with carbonyldiimidazole and methylamine or methylamine hydrochloride to produce a compound with the structure:

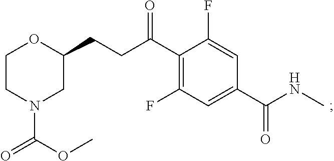 [SMILES: CNC(=O)c1cc(F)c(C(=O)CC[C@H]2CN(C(=O)OC)CCO2)c(F)c1]

G) followed b the reaction of the compound with the structure:

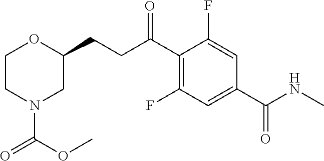 [SMILES: CNC(=O)c1cc(F)c(C(=O)CC[C@H]2CN(C(=O)OC)CCO2)c(F)c1]

with N-bromosuccinimide and trifluoromethanesulfonic acid to produce a compound with the structure:

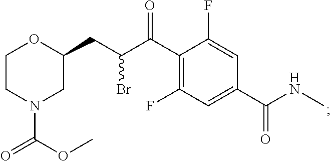 [SMILES: CNC(=O)c1cc(F)c(C(=O)C(Br)C[C@H]2CN(C(=O)OC)CCO2)c(F)c1]

H) followed by the reaction of the compound with the structure:

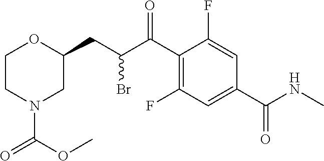 [SMILES: CNC(=O)c1cc(F)c(C(=O)C(Br)C[C@H]2CN(C(=O)OC)CCO2)c(F)c1]

with 4-methylpyridin-2-amine to produce a compound with the structure:

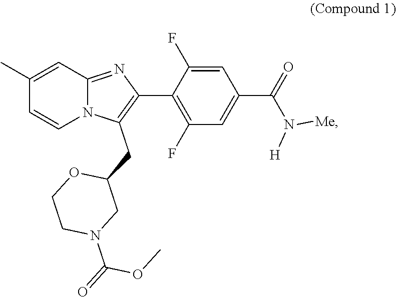 [SMILES: CNC(=O)c1cc(F)c(-c2nc3cc(C)ccn3c2C[C@H]2CN(C(=O)OC)CCO2)c(F)c1]

In another aspect described herein is a process for the preparation of methyl (S)-2-((2-(2,6-difluoro-4-(methylcarbamoyl)phenyl)-7-methylimidazo[1,2-a]pyridin-3-yl)methyl)morpholine-4-carboxylate (Compound 1), comprising:
A) the reaction of a compound with the structure:

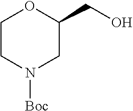 [SMILES: CC(C)(C)OC(=O)N1CCO[C@@H](CO)C1]

with toluenesulfonyl chloride and triethylamine to produce a compound with the structure:

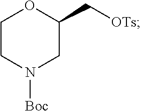 [SMILES: Cc1ccc(S(=O)(=O)OC[C@H]2CN(C(=O)OC(C)(C)C)CCO2)cc1]

B) followed by the reaction of the compound with the structure:

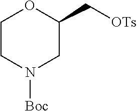 [SMILES: Cc1ccc(S(=O)(=O)OC[C@H]2CN(C(=O)OC(C)(C)C)CCO2)cc1]

with dimethylmalonate, potassium iodide, and cesium carbonate to produce a compound with the structure:

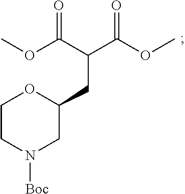 [SMILES: COC(=O)C(C[C@H]1CN(C(=O)OC(C)(C)C)CCO1)C(=O)OC]

C) followed by the reaction of the compounds with the structures:

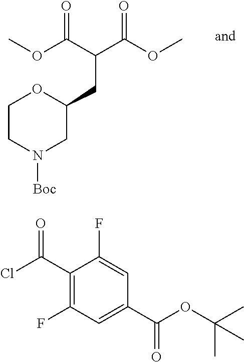

with triethylamine/magnesium chloride to produce a compound with the structure:

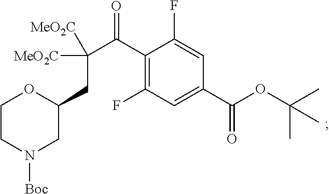 [SMILES: COC(=O)C(C[C@H]1CN(C(=O)OC(C)(C)C)CCO1)(C(=O)OC)C(=O)c1c(F)cc(C(=O)OC(C)(C)C)cc1F]

D) followed by the reaction of the compound with the structure:

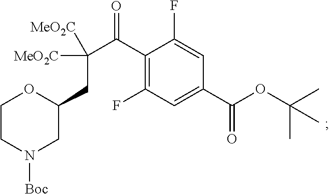 [SMILES: COC(=O)C(C[C@H]1CN(C(=O)OC(C)(C)C)CCO1)(C(=O)OC)C(=O)c1c(F)cc(C(=O)OC(C)(C)C)cc1F]

with lithium chloride and acetic acid to produce a compound with the structure;

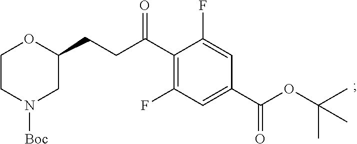 [SMILES: CC(C)(C)OC(=O)c1cc(F)c(C(=O)CC[C@H]2CN(C(=O)OC(C)(C)C)CCO2)c(F)c1]

E) followed by the reaction of the compound with the structure:

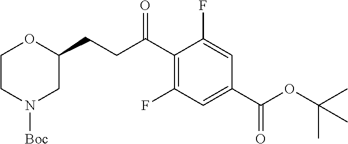 [SMILES: CC(C)(C)OC(=O)c1cc(F)c(C(=O)CC[C@H]2CN(C(=O)OC(C)(C)C)CCO2)c(F)c1]

with aqueous sodium hydroxide to produce a compound with the structure:

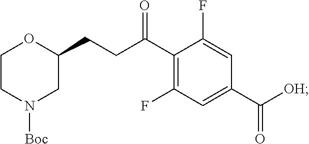 [SMILES: CC(C)(C)OC(=O)N1CCO[C@@H](CCC(=O)c2c(F)cc(C(=O)O)cc2F)C1]

F) followed by the reaction of the compound with the structure:

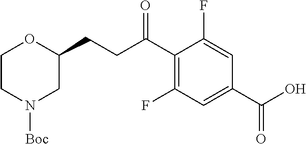 [SMILES: CC(C)(C)OC(=O)N1CCO[C@@H](CCC(=O)c2c(F)cc(C(=O)O)cc2F)C1]

with hydrogen chloride in ethyl acetate to produce a compound with the structure;

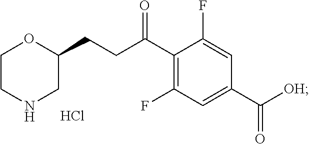 [SMILES: O=C(O)c1cc(F)c(C(=O)CC[C@H]2CNCCO2)c(F)c1 ; Cl]

G) followed by the reaction of the compound with the structure:

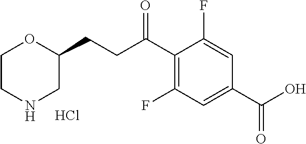 [SMILES: O=C(O)c1cc(F)c(C(=O)CC[C@H]2CNCCO2)c(F)c1 ; Cl]

with methyl chloroformate and aqueous sodium bicarbonate to produce a compound with the structure:

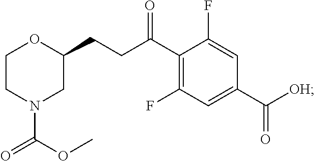 [SMILES: COC(=O)N1CCO[C@@H](CCC(=O)c2c(F)cc(C(=O)O)cc2F)C1]

H) followed by the reaction of the compound with the structure:

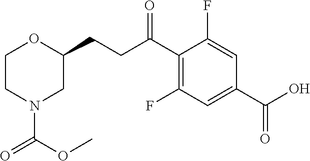 [SMILES: COC(=O)N1CCO[C@@H](CCC(=O)c2c(F)cc(C(=O)O)cc2F)C1]

with carbonyldiimidazole and methylamine or methylamine hydrochloride to produce a compound with the structure:

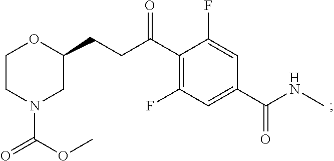 [SMILES: CNC(=O)c1cc(F)c(C(=O)CC[C@H]2CN(C(=O)OC)CCO2)c(F)c1]

I) followed by the reaction of the compound with the structure:

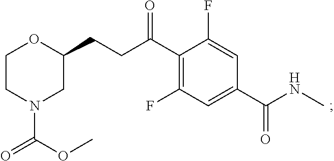 [SMILES: CNC(=O)c1cc(F)c(C(=O)CC[C@H]2CN(C(=O)OC)CCO2)c(F)c1]

with N-bromosuccinimide and trifluoromethanesulfonic acid to produce a compound with the structure:

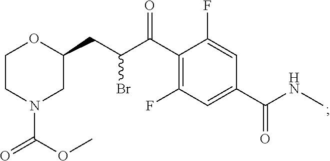 [SMILES: CNC(=O)c1cc(F)c(C(=O)C(Br)C[C@H]2CN(C(=O)OC)CCO2)c(F)c1]

J) followed by the reaction of the compound with the structure:

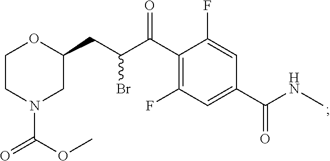 [SMILES: CNC(=O)c1cc(F)c(C(=O)C(Br)C[C@H]2CN(C(=O)OC)CCO2)c(F)c1]

with 4-methylpyridin-2-amine to produce a compound with the structure:

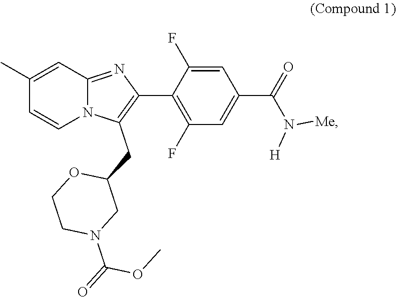 [SMILES: CNC(=O)c1cc(F)c(-c2nc3cc(C)ccn3c2C[C@H]2CN(C(=O)OC)CCO2)c(F)c1]

In another aspect described herein is a process for the preparation of methyl (S)-2-((2-(2,6-difluoro-4-(methylcarbamoyl)phenyl)-7-methylimidazo[1,2-a]pyridin-3-yl)methyl)morpholine-4-carboxylate (Compound 1):

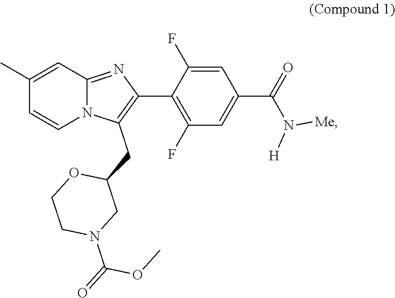 [SMILES: CNC(=O)c1cc(F)c(-c2nc3cc(C)ccn3c2C[C@H]2CN(C(=O)OC)CCO2)c(F)c1]

comprising contacting a compound with the structure:

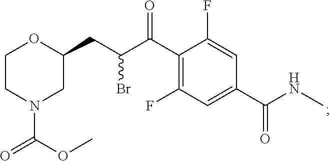 [SMILES: CNC(=O)c1cc(F)c(C(=O)C(Br)C[C@H]2CN(C(=O)OC)CCO2)c(F)c1]

with 4-methylpyridin-2-amine in the presence of a solvent. In some embodiments, the solvent is selected from acetonitrile and acetonitrile containing water.

In some embodiments of a process for the preparation of methyl (S)-2-((2-(2,6-difluoro-4-(methylcarbamoyl)phenyl)-7-methylimidazo[1,2-a]pyridin-3-yl)methyl)morpholine-4-carboxylate (Compound 1), the compound with the structure:

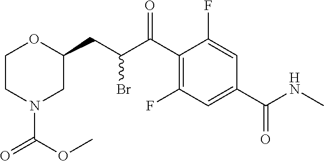 [SMILES: CNC(=O)c1cc(F)c(C(=O)C(Br)C[C@H]2CN(C(=O)OC)CCO2)c(F)c1]

is prepared by a process comprising contacting a compound with the structure:

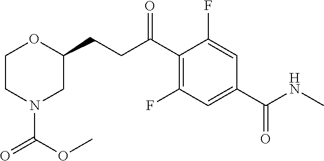 [SMILES: CNC(=O)c1cc(F)c(C(=O)CC[C@H]2CN(C(=O)OC)CCO2)c(F)c1]

with a brominating reagent. In some embodiments, the brominating agent is N-bromosuccinimide. In some embodiments, the brominating agent is CuBr₂. In some embodiments, the brominating agent is N-bromosuccinimide in the presence of acid. In some embodiments, the acid is trifluoromethanesulfonic acid. In some embodiments, the preparation of methyl (S)-2-((2-(2,6-difluoro-4-(methylcarbamoyl)phenyl)-7-methylimidazo[1,2-a]pyridin-3-yl)methyl)morpholine-4-carboxylate (Compound 1), the compound with the structure:

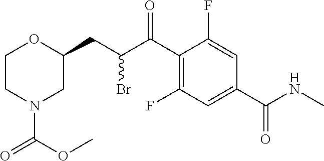 [SMILES: CNC(=O)c1cc(F)c(C(=O)C(Br)C[C@H]2CN(C(=O)OC)CCO2)c(F)c1]

is prepared by a process comprising contacting a compound with the structure:

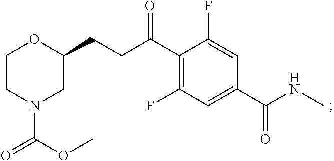 [SMILES: CNC(=O)c1cc(F)c(C(=O)CC[C@H]2CN(C(=O)OC)CCO2)c(F)c1]

with a brominating reagent in the presence of a solvent. In some embodiments, the solvent is DCM.

In some embodiments of a process for the preparation of methyl (S)-2-((2-(2,6-difluoro-4-(methylcarbamoyl)phenyl)-7-methylimidazo[1,2-a]pyridin-3-yl)methyl)morpholine-4-carboxylate (Compound 1), the compound with the structure:

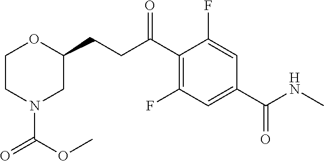 [SMILES: CNC(=O)c1cc(F)c(C(=O)CC[C@H]2CN(C(=O)OC)CCO2)c(F)c1]

is prepared by a process comprising contacting a compound with the structure:

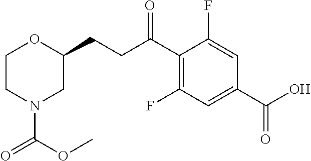 [SMILES: COC(=O)N1CCO[C@@H](CCC(=O)c2c(F)cc(C(=O)O)cc2F)C1]

with an amide coupling reagent and methylamine or a salt of methylamine. In some embodiments, the amide coupling reagent is carbonyldiimidazole. In some embodiments, the amide coupling reagent is propanephosphonic acid anhydride (T3P). In some embodiments, the preparation of methyl (S)-2-((2-(2,6-difluoro-4-(methylcarbamoyl)phenyl)-7-methylimidazo[1,2-a]pyridin-3-yl)methyl)morpholine-4-carboxylate (Compound 1), the compound with the structure:

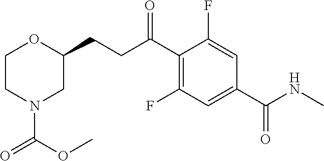 [SMILES: CNC(=O)c1cc(F)c(C(=O)CC[C@H]2CN(C(=O)OC)CCO2)c(F)c1]

is prepared by a process comprising contacting a compound with the structure:

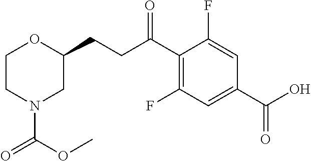 [SMILES: COC(=O)N1CCO[C@@H](CCC(=O)c2c(F)cc(C(=O)O)cc2F)C1]

with an amide coupling reagent and methylamine or a salt of methylamine, in the presence of a solvent and optionally in the presence of a base. In some embodiments, the solvent is THF. In some embodiments, the base is N,N-diisopropylethylamine (DIPEA).

In some embodiments of a process for the preparation of methyl (S)-2-((2-(2,6-difluoro-4-(methylcarbamoyl)phenyl)-7-methylimidazo[1,2-a]pyridin-3-yl)methyl)morpholine-4-carboxylate (Compound 1), the compound with the structure:

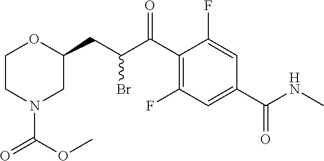 [SMILES: CNC(=O)c1cc(F)c(C(=O)C(Br)C[C@H]2CN(C(=O)OC)CCO2)c(F)c1]

is prepared by a process comprising contacting a compound with the structure:

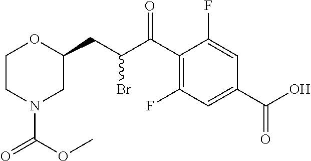 [SMILES: COC(=O)N1CCO[C@@H](CC(Br)C(=O)c2c(F)cc(C(=O)O)cc2F)C1]

with an amide coupling reagent and methylamine or a salt of methylamine. In some embodiments, the amide coupling reagent is carbonyldiimidazole. In some embodiments, the amide coupling reagent is propanephosphonic acid anhydride (T3P).

In some embodiments of a process for the preparation of methyl (S)-2-((2-(2,6-difluoro-4-(methylcarbamoyl)phenyl)-7-methylimidazo[1,2-a]pyridin-3-yl)methyl)morpholine-4-carboxylate (Compound 1), the compound with the structure:

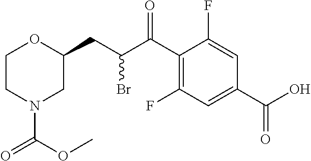 [SMILES: COC(=O)N1CCO[C@@H](CC(Br)C(=O)c2c(F)cc(C(=O)O)cc2F)C1]

is prepared by a process comprising contacting a compound with the structure:

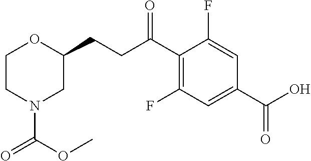 [SMILES: COC(=O)N1CCO[C@@H](CCC(=O)c2c(F)cc(C(=O)O)cc2F)C1]

with a brominating agent. In some embodiments, the brominating agent is N-bromosuccinimide. In some embodiments, the brominating agent is CuBr₂. In some embodiments, the brominating agent is N-bromosuccinimide in the presence of acid. In some embodiments, the acid is trifluoromethanesulfonic acid.

In some embodiments of a process for the preparation of methyl (S)-2-((2-(2,6-difluoro-4-(methylcarbamoyl)phenyl)-7-methylimidazo[1,2-a]pyridin-3-yl)methyl)morpholine-4-carboxylate (Compound 1), the compound with the structure:

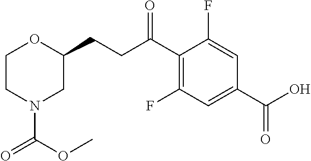 [SMILES: COC(=O)N1CCO[C@@H](CCC(=O)c2c(F)cc(C(=O)O)cc2F)C1]

is prepared by a process comprising contacting a compound with the structure

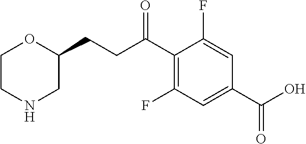 [SMILES: O=C(O)c1cc(F)c(C(=O)CC[C@H]2CNCCO2)c(F)c1]

with methyl chloroformate and a base. In some embodiments, the base is aqueous sodium bicarbonate.

In some embodiments of a process for the preparation of methyl (S)-2-((2-(2,6-difluoro-4-(methylcarbamoyl)phenyl)-7-methylimidazo[1,2-a]pyridin-3-yl)methyl)morpholine-4-carboxylate (Compound 1), the compound with the structure:

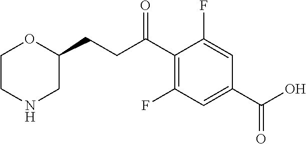 [SMILES: O=C(O)c1cc(F)c(C(=O)CC[C@H]2CNCCO2)c(F)c1]

is prepared by a process comprising contacting a compound with the structure:

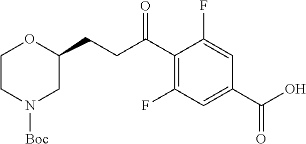 [SMILES: CC(C)(C)OC(=O)N1CCO[C@@H](CCC(=O)c2c(F)cc(C(=O)O)cc2F)C1]

with a deprotection reagent selected from 1) hydrogen chloride in the presence of a solvent, 2) trifluoroacetic acid, and 3) phosphoric acid. In some embodiments, the deprotection reagent is hydrogen chloride in the presence of a solvent and the solvent is ethyl acetate. In some embodiments, the deprotection reagent is trifluoroacetic acid. In some embodiments, the deprotection reagent is) phosphoric acid.

In some embodiments of a process for the preparation of methyl (S)-2-((2-(2,6-difluoro-4-(methylcarbamoyl)phenyl)-7-methylimidazo[1,2-a]pyridin-3-yl)methyl)morpholine-4-carboxylate (Compound 1), the compound with the structure:

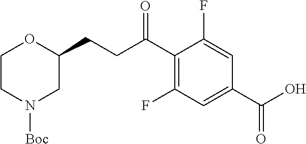 [SMILES: CC(C)(C)OC(=O)N1CCO[C@@H](CCC(=O)c2c(F)cc(C(=O)O)cc2F)C1]

is prepared by a process comprising contacting a compound with the structure:

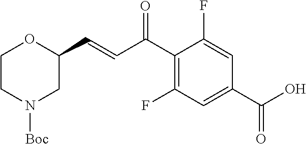 [SMILES: CC(C)(C)OC(=O)N1CCO[C@@H](/C=C/C(=O)c2c(F)cc(C(=O)O)cc2F)C1]

with a hydrogenation catalyst and hydrogen. In some embodiments, the hydrogenation catalyst is palladium on carbon, palladium hydroxide, rhodium on carbon, rhodium on alumina, platinum oxide, or platinum on carbon. In some embodiments, the hydrogenation catalyst is palladium on carbon.

In some embodiments of a process for the preparation of methyl (S)-2-((2-(2,6-difluoro-4-(methylcarbamoyl)phenyl)-7-methylimidazo[1,2-a]pyridin-3-yl)methyl)morpholine-4-carboxylate (Compound 1), the compound with the structure:

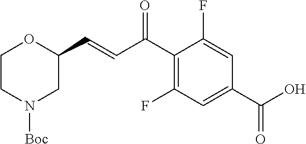 [SMILES: CC(C)(C)OC(=O)N1CCO[C@@H](/C=C/C(=O)c2c(F)cc(C(=O)O)cc2F)C1]

is prepared by a process comprising contacting a compound with the structure:

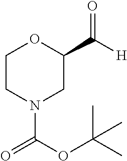

with a compound with the structure:

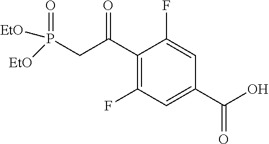 [SMILES: CCOP(=O)(CC(=O)c1c(F)cc(C(=O)O)cc1F)OCC]

with a base. In some embodiments, the base is a mixture of aqueous potassium bicarbonate and potassium carbonate. In some embodiments, the base is potassium phosphate.

In some embodiments of a process for the preparation of methyl (S)-2-((2-(2,6-difluoro-4-(methylcarbamoyl)phenyl)-7-methylimidazo[1,2-a]pyridin-3-yl)methyl)morpholine-4-carboxylate (Compound 1), the compound with the structure:

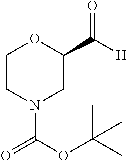

is prepared by a process comprising contacting a compound with the structure:

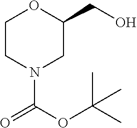

with an oxidizing agent. In some embodiments, the oxidizing agent is selected from 2,2,6,6-tetramethylpiperidine 1-oxyl, T3P, and trichloroisocyanuric acid (TCCA). In some embodiments, the oxidizing agent is 2,2,6,6-tetramethylpiperidine 1-oxyl. In some embodiments, the oxidizing agent is T3P. In some embodiments, the oxidizing agent is trichloroisocyanuric acid (TCCA).

In some embodiments of a process for the preparation of methyl (S)-2-((2-(2,6-difluoro-4-(methylcarbamoyl)phenyl)-7-methylimidazo[1,2-a]pyridin-3-yl)methyl)morpholine-4-carboxylate (Compound 1), the compound with the structure:

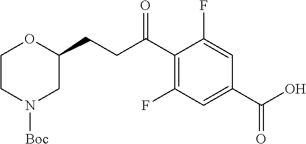 [SMILES: CC(C)(C)OC(=O)N1CCO[C@@H](CCC(=O)c2c(F)cc(C(=O)O)cc2F)C1]

is prepared by a process comprising contacting a compound with the structure:

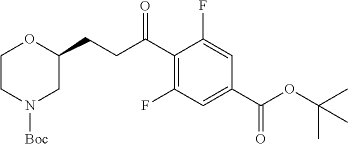 [SMILES: CC(C)(C)OC(=O)c1cc(F)c(C(=O)CC[C@H]2CN(C(=O)OC(C)(C)C)CCO2)c(F)c1]

with a base in a solvent. In some embodiments, the base is aqueous sodium hydroxide. In some embodiments, the solvent is selected from tetrahydrofuran, N-methyl-2-pyrrolidone, methanol, isopropanol, and tert-butanol. In some embodiments, the solvent is isopropanol.

In some embodiments of a process for the preparation of methyl (S)-2-((2-(2,6-difluoro-4-(methylcarbamoyl)phenyl)-7-methylimidazo[1,2-a]pyridin-3-yl)methyl)morpholine-4-carboxylate (Compound 1), the compound with the structure:

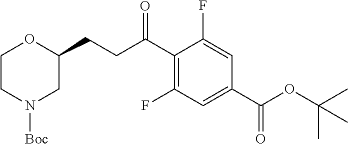 [SMILES: CC(C)(C)OC(=O)c1cc(F)c(C(=O)CC[C@H]2CN(C(=O)OC(C)(C)C)CCO2)c(F)c1]

is prepared by a process comprising contacting a compound with the structure:

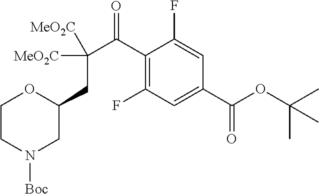 [SMILES: COC(=O)C(C[C@H]1CN(C(=O)OC(C)(C)C)CCO1)(C(=O)OC)C(=O)c1c(F)cc(C(=O)OC(C)(C)C)cc1F]

with lithium chloride and acetic acid.

In some embodiments of a process for the preparation of methyl (S)-2-((2-(2,6-difluoro-4-(methylcarbamoyl)phenyl)-7-methylimidazo[1,2-a]pyridin-3-yl)methyl)morpholine-4-carboxylate (Compound 1), the compound with the structure:

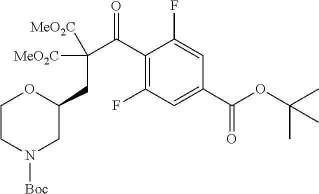 [SMILES: COC(=O)C(C[C@H]1CN(C(=O)OC(C)(C)C)CCO1)(C(=O)OC)C(=O)c1c(F)cc(C(=O)OC(C)(C)C)cc1F]

is prepared by a process comprising contacting a compound with the structure:

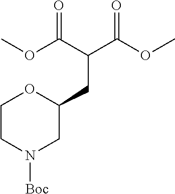 [SMILES: COC(=O)C(C[C@H]1CN(C(=O)OC(C)(C)C)CCO1)C(=O)OC]

with a compound with the structure:

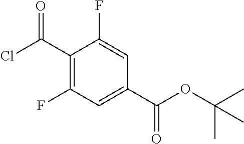 [SMILES: CC(C)(C)OC(=O)c1cc(F)c(C(=O)Cl)c(F)c1]

and a base, in the presence of a solvent. In some embodiments, the base is selected from sodium hydride, lithium bis(trimethylsilyl)amide, potassium tert-butoxide, and triethylamine/magnesium chloride. In some embodiments, the base is triethylamine/magnesium chloride. In some embodiments, the solvent is tetrahydrofuran or 2-methyltetrahydrofuran. In some embodiments, the solvent is 2-methyltetrahydrofuran.

In some embodiments of a process for the preparation of methyl (S)-2-((2-(2,6-difluoro-4-(methylcarbamoyl)phenyl)-7-methylimidazo[1,2-a]pyridin-3-yl)methyl)morpholine-4-carboxylate (Compound 1), the compound with the structure:

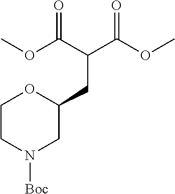 [SMILES: COC(=O)C(C[C@H]1CN(C(=O)OC(C)(C)C)CCO1)C(=O)OC]

is prepared by a process comprising contacting a compound with the structure:

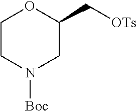 [SMILES: Cc1ccc(S(=O)(=O)OC[C@H]2CN(C(=O)OC(C)(C)C)CCO2)cc1]

with dimethylmalonate, potassium iodide, and a base. In some embodiments, the base is selected from sodium hydride, lithium bis(trimethylsilyl)amide, sodium bis(trimethylsilyl)amide, potassium bis(trimethylsilyl)amide, potassium carbonate, sodium carbonate, and cesium carbonate. In some embodiments, the base is cesium carbonate.

In some embodiments of a process for the preparation of methyl (S)-2-((2-(2,6-difluoro-4-(methylcarbamoyl)phenyl)-7-methylimidazo[1,2-a]pyridin-3-yl)methyl)morpholine-4-carboxylate (Compound 1), the compound with the structure:

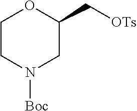 [SMILES: Cc1ccc(S(=O)(=O)OC[C@H]2CN(C(=O)OC(C)(C)C)CCO2)cc1]

is prepared by a process comprising contacting a compound with the structure:

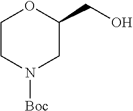 [SMILES: CC(C)(C)OC(=O)N1CCO[C@@H](CO)C1]

with toluenesulfonyl chloride and a base in the presence of a solvent. In some embodiments, the base is triethylamine. In some embodiments, the solvent is dichloromethane.

Further disclosed herein is a compound having the structure:

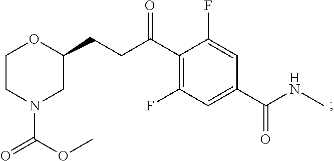 [SMILES: CNC(=O)c1cc(F)c(C(=O)CC[C@H]2CN(C(=O)OC)CCO2)c(F)c1]

or a pharmaceutically acceptable salt or co-crystal thereof.

Further disclosed herein is a compound having the structure:

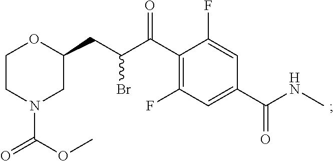 [SMILES: CNC(=O)c1cc(F)c(C(=O)C(Br)C[C@H]2CN(C(=O)OC)CCO2)c(F)c1]

or a pharmaceutically acceptable salt or co-crystal thereof.

Further disclosed herein is a compound having the structure:

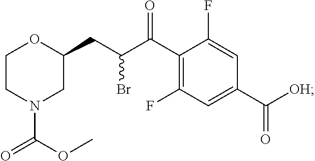 [SMILES: COC(=O)N1CCO[C@@H](CC(Br)C(=O)c2c(F)cc(C(=O)O)cc2F)C1]

or a pharmaceutically acceptable salt thereof.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE INVENTION

Good manufacturing practices are usually required for large scale manufacture of clinically useful drug candidates. Provided herein are certain processes and methods for the manufacture of P2X3 antagonist methyl (S)-2-((2-(2,6-difluoro-4-(methylcarbamoyl)phenyl)-7-methylimidazo[1,2-a]pyridin-3-yl)methyl)morpholine-4-carboxylate (Compound 1), or a pharmaceutically acceptable salt or co-crystal thereof.

### Definitions

As used in the specification and appended claims, unless specified to the contrary, the following terms have the meaning indicated below.

As used herein and in the appended claims, the singular forms “a,” “and,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof.

When ranges are used herein for physical properties, such as molecular weight, or chemical properties, such as chemical formulae, all combinations and subcombinations of ranges and specific embodiments therein are intended to be included.

The term “about” when referring to a number or a numerical range means that the number or numerical range referred to is an approximation within experimental variability (or within statistical experimental error), and thus the number or numerical range varies between 1% and 15% of the stated number or numerical range.

The term “comprising” (and related terms such as “comprise” or “comprises” or “having” or “including”) is not intended to exclude that which in other certain embodiments, for example, an embodiment of any composition of matter, composition, method, or process, or the like, described herein, “consist of” or “consist essentially of” the described features.

The term “subject” or “patient” encompasses mammals and non-mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods and compositions provided herein, the mammal is a human.

As used herein, “treatment” or “treating” or “palliating” or “ameliorating” are used interchangeably herein. These terms refers to an approach for obtaining beneficial or desired results including but not limited to therapeutic benefit and/or a prophylactic benefit. By “therapeutic benefit” is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient is still afflicted with the underlying disorder. For prophylactic benefit, the compositions are administered to a patient at risk of developing a particular disease, or to a patient reporting one or more of the physiological symptoms of a disease, even though a diagnosis of this disease has been made.

“Pharmaceutically acceptable salt” includes both acid and base addition salts. A pharmaceutically acceptable salt of any one of the compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and, aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997)). Acid addition salts of basic compounds are prepared by contacting the free base forms with a sufficient amount of the desired acid to produce the salt.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. In some embodiments, pharmaceutically acceptable base addition salts are formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, NN-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

The term “pharmaceutical combination” as used herein, means a product that results from the mixing or combining of more than one active ingredient and includes both fixed and non-fixed combinations of the active ingredients. The term “fixed combination” means that the active ingredients, are both administered to a patient simultaneously in the form of a single entity or dosage. The term “non-fixed combination” means that the active ingredients, are administered to a patient as separate entities either simultaneously, concurrently or sequentially with no specific intervening time limits, wherein such administration provides effective levels of the two compounds in the body of the patient. The latter also applies to cocktail therapy, e.g. the administration of three or more active ingredients.

The terms “co-administration” or the like, as used herein, are meant to encompass administration of the selected therapeutic agents to a single patient, and are intended to include treatment regimens in which the agents are administered by the same or different route of administration or at the same or different time.

The term “activator” is used in this specification to denote any molecular species that results in activation of the indicated receptor, regardless of whether the species itself binds to the receptor or a metabolite of the species binds to the receptor when the species is administered topically. Thus, the activator can be a ligand of the receptor or it can be an activator that is metabolized to the ligand of the receptor, i.e., a metabolite that is formed in tissue and is the actual ligand.

The term “antagonist” as used herein, refers to a small-molecule agent that binds to a nuclear hormone receptor and subsequently decreases the agonist induced transcriptional activity of the nuclear hormone receptor.

The term “agonist” as used herein, refers to a small-molecule agent that binds to a nuclear hormone receptor and subsequently increases nuclear hormone receptor transcriptional activity in the absence of a known agonist.

The term “inverse agonist” as used herein, refers to a small-molecule agent that binds to a nuclear hormone receptor and subsequently decreases the basal level of nuclear hormone receptor transcriptional activity that is present in the absence of a known agonist.

The term “modulate,” as used herein, means to interact with a target protein either directly or indirectly so as to alter the activity of the target protein, including, by way of example only, to inhibit the activity of the target, or to limit or reduce the activity of the target.

As used herein, the term “modulator” refers to a compound that alters an activity of a target. For example, a modulator can cause an increase or decrease in the magnitude of a certain activity of a target compared to the magnitude of the activity in the absence of the modulator. In certain embodiments, a modulator is an inhibitor, which decreases the magnitude of one or more activities of a target. In certain embodiments, an inhibitor completely prevents one or more activities of a target.

### Compounds

In some embodiments, the P2X3 antagonist described herein is methyl (S)-2-((2-(2,6-difluoro-4-(methylcarbamoyl)phenyl)-7-methylimidazo[1,2-a]pyridin-3-yl)methyl)morpholine-4-carboxylate (Compound 1), or a pharmaceutically acceptable salt or co-crystal thereof. Compound 1 has the structure:

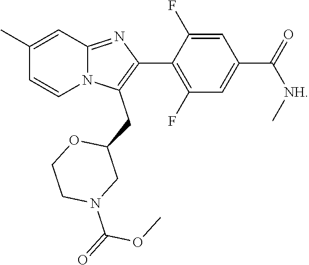 [SMILES: CNC(=O)c1cc(F)c(-c2nc3cc(C)ccn3c2C[C@H]2CN(C(=O)OC)CCO2)c(F)c1]

In some embodiments, an intermediate in the synthesis of Compound 1 is

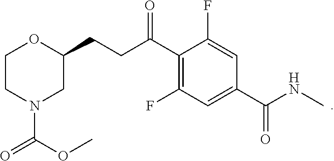 [SMILES: CNC(=O)c1cc(F)c(C(=O)CC[C@H]2CN(C(=O)OC)CCO2)c(F)c1]

In some embodiments, an intermediate in the synthesis of Compound 1 is

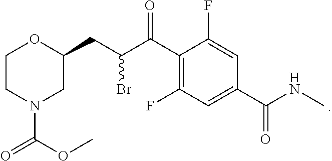 [SMILES: CNC(=O)c1cc(F)c(C(=O)C(Br)C[C@H]2CN(C(=O)OC)CCO2)c(F)c1]

In some embodiments, an intermediate in the synthesis of Compound 1 is

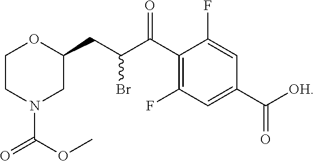 [SMILES: COC(=O)N1CCO[C@@H](CC(Br)C(=O)c2c(F)cc(C(=O)O)cc2F)C1]

### Further Forms of Compounds

The compounds described herein may in some cases exist as diastereomers, enantiomers, or other stereoisomeric forms. The compounds presented herein include all diastereomeric, enantiomeric, and epimeric forms as well as the appropriate mixtures thereof. Separation of stereoisomers may be performed by chromatography or by the forming diastereomeric and separation by recrystallization, or chromatography, or any combination thereof. (Jean Jacques, Andre Collet, Samuel H. Wilen, “Enantiomers, Racemates and Resolutions”, John Wiley And Sons, Inc., 1981, herein incorporated by reference for this disclosure). Stereoisomers may also be obtained by stereoselective synthesis.

In some situations, compounds may exist as tautomers. All tautomers are included within the formulas described herein.

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefore react with any of a number of inorganic or organic bases, and inorganic and organic acids, to form a pharmaceutically acceptable salt. In some embodiments, these salts are prepared in situ during the final isolation and purification of the compounds of the invention, or by separately reacting a purified compound in its free form with a suitable acid or base, and isolating the salt thus formed.

In some embodiments, the pharmaceutically acceptable salt of Compound 1 is an acetate, benzoate, besylate, bitartrate, carbonate, citrate, fumarate, gluconate, hydrobromide, hydrochloride, maleate, mesylate, nitrate, phosphate, salicylate, succinate, sulfate, or tartrate salt. In some embodiments, the pharmaceutically acceptable salt of Compound 1 is a mono-hydrochloride salt. In further embodiments, the pharmaceutically acceptable salt of Compound 1 is a mono-hydrochloride salt.

### Solvates

In some embodiments, the compounds described herein exist as solvates. The invention provides for methods of treating diseases by administering such solvates. The invention further provides for methods of treating diseases by administering such solvates as pharmaceutical compositions.

Solvates contain either stoichiometric or non-stoichiometric amounts of a solvent, and, in some embodiments, are formed during the process of crystallization with pharmaceutically acceptable solvents such as water, ethanol, and the like. Hydrates are formed when the solvent is water, or alcoholates are formed when the solvent is alcohol. Solvates of the compounds described herein are conveniently prepared or formed during the processes described herein. By way of example only, hydrates of the compounds described herein are conveniently prepared by recrystallization from an aqueous/organic solvent mixture, using organic solvents including, but not limited to, dioxane, tetrahydrofuran or methanol. In addition, the compounds provided herein exist in unsolvated as well as solvated forms. In general, the solvated forms are considered equivalent to the unsolvated forms for the purposes of the compounds and methods provided herein.

### Labeled Compounds

In some embodiments, the compounds described herein exist in their isotopically-labeled forms. In some embodiments, the methods disclosed herein include methods of treating diseases by administering such isotopically-labeled compounds. In some embodiments, the methods disclosed herein include methods of treating diseases by administering such isotopically-labeled compounds as pharmaceutical compositions. Thus, in some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. Examples of isotopes that are incorporated into compounds of the invention include isotopes of hydrogen, carbon, nitrogen, oxygen, phosphorous, sulfur, fluorine and chloride, such as ²H, ³H, ¹C, ¹⁴C, ¹⁵N, ¹⁸O, ¹⁷O, ³¹P, ³²P, ³⁵S, ¹⁸F, and ³⁶Cl, respectively. Compounds described herein, and pharmaceutically acceptable salts, esters, solvate, hydrates or derivatives thereof which contain the aforementioned isotopes and/or other isotopes of other atoms are within the scope of this invention. Certain isotopically-labeled compounds, for example those into which radioactive isotopes such as ³H and ¹⁴C are incorporated, are useful in drug and/or substrate tissue distribution assays. Tritiated, i. e., ³H and carbon-14, i. e., ¹⁴C, isotopes are particularly preferred for their ease of preparation and detectability. Further, substitution with heavy isotopes such as deuterium, i.e., ²H, produces certain therapeutic advantages resulting from greater metabolic stability, for example increased in vivo half-life or reduced dosage requirements. Increased levels of deuterium incorporation produce a detectable kinetic isotope effect (KIE) that may affect the pharmacokinetic, pharmacologic and/or toxicologic parameters of Compound 1 in comparison to Compound 1 having naturally occurring levels of deuterium. In some embodiments, the isotopically labeled compound, or a pharmaceutically acceptable salt thereof, is prepared by any suitable method.

In some embodiments, at least one hydrogen in Compound 1 is replaced with deuterium.

In some embodiments, the compounds described herein are labeled by other means, including, but not limited to, the use of chromophores or fluorescent moieties, bioluminescent labels, or chemiluminescent labels.

### Process for Preparation

In some embodiments, the synthesis of compounds described herein are accomplished using means described in the chemical literature, using the methods described herein, or by a combination thereof. In addition, solvents, temperatures and other reaction conditions presented herein may vary.

In other embodiments, the starting materials and reagents used for the synthesis of the compounds described herein are synthesized or are obtained from commercial sources, such as, but not limited to, Sigma-Aldrich, FischerScientific (Fischer Chemicals), and AcrosOrganics.

In further embodiments, the compounds described herein, and other related compounds having different substituents are synthesized using techniques and materials described herein as well as those that are recognized in the field, such as described, for example, in Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey and Sundberg, Advanced Organic Chemistry 4^{th} Ed., Vols. A and B (Plenum 2000, 2001), and Green and Wuts, Protective Groups in Organic Synthesis 3^{rd} Ed., (Wiley 1999) (all of which are incorporated by reference for such disclosure). General methods for the preparation of compounds as disclosed herein may be derived from reactions and the reactions may be modified by the use of appropriate reagents and conditions, for the introduction of the various moieties found in the formulae as provided herein.

In some embodiments is a process for the preparation of methyl (S)-2-((2-(2,6-difluoro-4-(methylcarbamoyl)phenyl)-7-methylimidazo[1,2-a]pyridin-3-yl)methyl)morpholine-4-carboxylate (Compound 1):

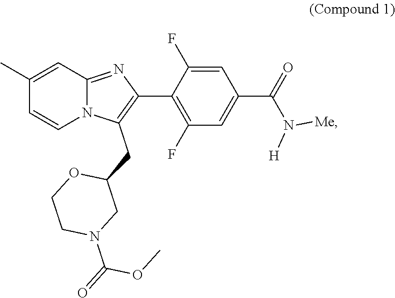 [SMILES: CNC(=O)c1cc(F)c(-c2nc3cc(C)ccn3c2C[C@H]2CN(C(=O)OC)CCO2)c(F)c1]

comprising contacting a compound with the structure:

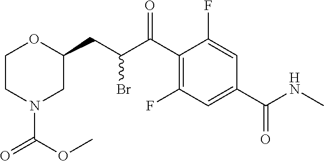 [SMILES: CNC(=O)c1cc(F)c(C(=O)C(Br)C[C@H]2CN(C(=O)OC)CCO2)c(F)c1]

with 4-methylpyridin-2-amine in the presence of a solvent. In some embodiments, the solvent is selected from acetonitrile and acetonitrile containing water. In some embodiments, the solvent is acetonitrile. In some embodiments, the solvent is acetonitrile containing water.

In some embodiments of a process for the preparation of methyl (S)-2-((2-(2,6-difluoro-4-(methylcarbamoyl)phenyl)-7-methylimidazo[1,2-a]pyridin-3-yl)methyl)morpholine-4-carboxylate (Compound 1), the compound with the structure:

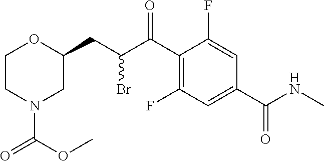 [SMILES: CNC(=O)c1cc(F)c(C(=O)C(Br)C[C@H]2CN(C(=O)OC)CCO2)c(F)c1]

is prepared by a process comprising contacting a compound with the structure:

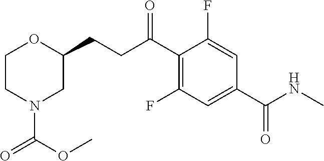 [SMILES: CNC(=O)c1cc(F)c(C(=O)CC[C@H]2CN(C(=O)OC)CCO2)c(F)c1]

with a brominating reagent. In some embodiments, the brominating agent is N-bromosuccinimide in the presence of acid. In some embodiments, the acid is trifluoromethanesulfonic acid. In some embodiments, the brominating agent is copper(II) bromide.

In some embodiments of a process for the preparation of methyl (S)-2-((2-(2,6-difluoro-4-(methylcarbamoyl)phenyl)-7-methylimidazo[1,2-a]pyridin-3-yl)methyl)morpholine-4-carboxylate (Compound 1), the compound with the structure:

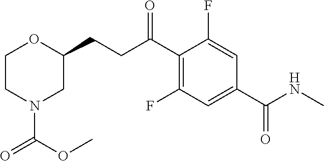 [SMILES: CNC(=O)c1cc(F)c(C(=O)CC[C@H]2CN(C(=O)OC)CCO2)c(F)c1]

is prepared by a process comprising contacting a compound with the structure:

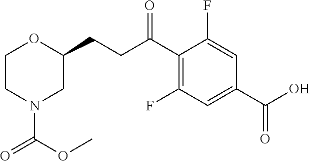 [SMILES: COC(=O)N1CCO[C@@H](CCC(=O)c2c(F)cc(C(=O)O)cc2F)C1]

with an amide coupling reagent and methylamine or a salt of methylamine. In some embodiments, the amide coupling reagent is carbonyldiimidazole. In some embodiments, the amide coupling reagent is propanephosphonic acid anhydride (T3P).

In some embodiments of a process for the preparation of methyl (S)-2-((2-(2,6-difluoro-4-(methylcarbamoyl)phenyl)-7-methylimidazo[1,2-a]pyridin-3-yl)methyl)morpholine-4-carboxylate (Compound 1), the compound with the structure:

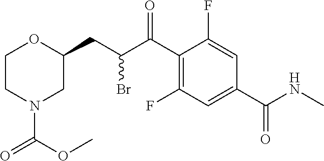 [SMILES: CNC(=O)c1cc(F)c(C(=O)C(Br)C[C@H]2CN(C(=O)OC)CCO2)c(F)c1]

is prepared by a process comprising contacting a compound with the structure:

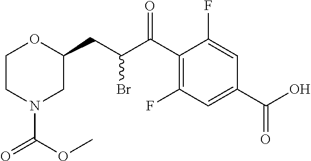 [SMILES: COC(=O)N1CCO[C@@H](CC(Br)C(=O)c2c(F)cc(C(=O)O)cc2F)C1]

with an amide coupling reagent and methylamine or a salt of methylamine. In some embodiments, the amide coupling reagent is carbonyldiimidazole. In some embodiments, the amide coupling reagent is propanephosphonic acid anhydride (T3P).

In some embodiments of a process for the preparation of methyl (S)-2-((2-(2,6-difluoro-4-(methylcarbamoyl)phenyl)-7-methylimidazo[1,2-a]pyridin-3-yl)methyl)morpholine-4-carboxylate (Compound 1), the compound with the structure:

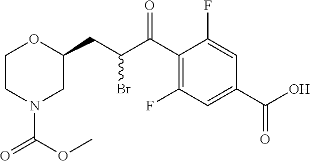 [SMILES: COC(=O)N1CCO[C@@H](CC(Br)C(=O)c2c(F)cc(C(=O)O)cc2F)C1]

is prepared by a process comprising contacting a compound with the structure:

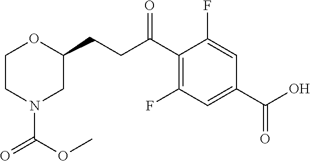 [SMILES: COC(=O)N1CCO[C@@H](CCC(=O)c2c(F)cc(C(=O)O)cc2F)C1]

with a brominating agent. In some embodiments, the brominating agent is N-bromosuccinimide in the presence of acid. In some embodiments, the acid is trifluoromethanesulfonic acid. In some embodiments, the brominating agent is copper(II) bromide. In some embodiments, the brominating agent is liquid bromine.

In some embodiments of a process for the preparation of methyl (S)-2-((2-(2,6-difluoro-4-(methylcarbamoyl)phenyl)-7-methylimidazo[1,2-a]pyridin-3-yl)methyl)morpholine-4-carboxylate (Compound 1), the compound with the structure:

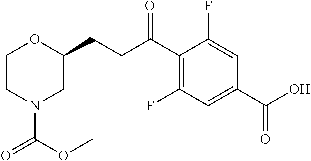 [SMILES: COC(=O)N1CCO[C@@H](CCC(=O)c2c(F)cc(C(=O)O)cc2F)C1]

is prepared by a process comprising contacting a compound with the structure:

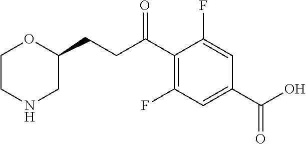 [SMILES: O=C(O)c1cc(F)c(C(=O)CC[C@H]2CNCCO2)c(F)c1]

with methyl chloroformate and a base. In some embodiments, the base is aqueous sodium bicarbonate.

In some embodiments of a process for the preparation of methyl (S)-2-((2-(2,6-difluoro-4-(methylcarbamoyl)phenyl)-7-methylimidazo[1,2-a]pyridin-3-yl)methyl)morpholine-4-carboxylate (Compound 1), the compound with the structure:

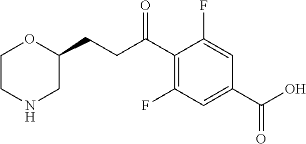 [SMILES: O=C(O)c1cc(F)c(C(=O)CC[C@H]2CNCCO2)c(F)c1]

is prepared by a process comprising contacting a compound with the structure:

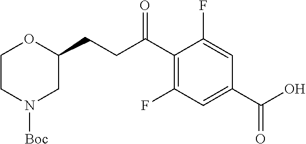 [SMILES: CC(C)(C)OC(=O)N1CCO[C@@H](CCC(=O)c2c(F)cc(C(=O)O)cc2F)C1]

with a deprotection reagent selected from 1) hydrogen chloride in the presence of a solvent, 2) trifluoroacetic acid, and 3) phosphoric acid. In some embodiments, the deprotection reagent is hydrogen chloride in the presence of a solvent and the solvent is ethyl acetate. In some embodiments, the deprotection reagent is trifluoroacetic acid. In some embodiments, the deprotection reagent is) phosphoric acid.

In some embodiments of a process for the preparation of methyl (S)-2-((2-(2,6-difluoro-4-(methylcarbamoyl)phenyl)-7-methylimidazo[1,2-a]pyridin-3-yl)methyl)morpholine-4-carboxylate (Compound 1), the compound with the structure:

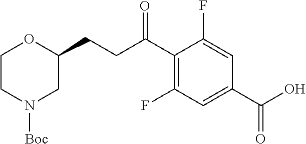 [SMILES: CC(C)(C)OC(=O)N1CCO[C@@H](CCC(=O)c2c(F)cc(C(=O)O)cc2F)C1]

is prepared by a process comprising contacting a compound with the structure:

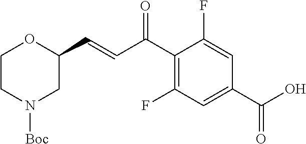 [SMILES: CC(C)(C)OC(=O)N1CCO[C@@H](/C=C/C(=O)c2c(F)cc(C(=O)O)cc2F)C1]

with a hydrogenation catalyst and hydrogen. In some embodiments, the hydrogenation catalyst is palladium on carbon, palladium hydroxide, rhodium on carbon, rhodium on alumina, platinum oxide, or platinum on carbon. In some embodiments, the hydrogenation catalyst is palladium on carbon. In some embodiments, the hydrogenation catalyst is palladium hydroxide. In some embodiments, the hydrogenation catalyst is rhodium on carbon. In some embodiments, the hydrogenation catalyst is rhodium on alumina. In some embodiments, the hydrogenation catalyst is platinum oxide. In some embodiments, the hydrogenation catalyst is platinum on carbon.

In some embodiments of a process for the preparation of methyl (S)-2-((2-(2,6-difluoro-4-(methylcarbamoyl)phenyl)-7-methylimidazo[1,2-a]pyridin-3-yl)methyl)morpholine-4-carboxylate (Compound 1), the compound with the structure:

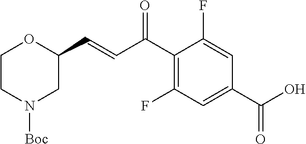 [SMILES: CC(C)(C)OC(=O)N1CCO[C@@H](/C=C/C(=O)c2c(F)cc(C(=O)O)cc2F)C1]

is prepared by a process comprising contacting a compound with the structure:

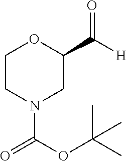

with a compound with the structure:

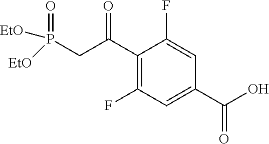 [SMILES: CCOP(=O)(CC(=O)c1c(F)cc(C(=O)O)cc1F)OCC]

with a base. In some embodiments, the base is a mixture of aqueous potassium bicarbonate and potassium carbonate. In some embodiments, the base is potassium bicarbonate. In some embodiments, the base is potassium carbonate. In some embodiments, the base is potassium phosphate.

In some embodiments of a process for the preparation of methyl (S)-2-((2-(2,6-difluoro-4-(methylcarbamoyl)phenyl)-7-methylimidazo[1,2-a]pyridin-3-yl)methyl)morpholine-4-carboxylate (Compound 1), the compound with the structure:

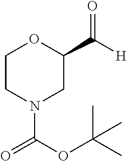

is prepared by a process comprising contacting a compound with the structure:

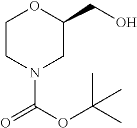

with an oxidizing agent. In some embodiments, the oxidizing agent is selected from 2,2,6,6-tetramethylpiperidine 1-oxyl, T3P, and trichloroisocyanuric acid (TCCA). In some embodiments, the oxidizing agent is 2,2,6,6-tetramethylpiperidine 1-oxyl. In some embodiments, the oxidizing agent is T3P. In some embodiments, the oxidizing agent is trichloroisocyanuric acid (TCCA).

In some embodiments of a process for the preparation of methyl (S)-2-((2-(2,6-difluoro-4-(methylcarbamoyl)phenyl)-7-methylimidazo[1,2-a]pyridin-3-yl)methyl)morpholine-4-carboxylate (Compound 1), the compound with the structure:

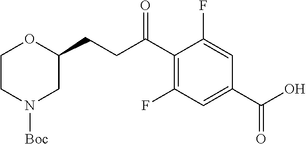 [SMILES: CC(C)(C)OC(=O)N1CCO[C@@H](CCC(=O)c2c(F)cc(C(=O)O)cc2F)C1]

is prepared by a process comprising contacting a compound with the structure:

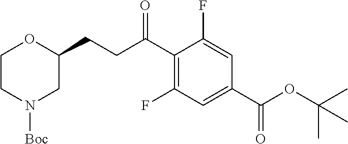 [SMILES: CC(C)(C)OC(=O)c1cc(F)c(C(=O)CC[C@H]2CN(C(=O)OC(C)(C)C)CCO2)c(F)c1]

with a base in a solvent. In some embodiments, the base is aqueous sodium hydroxide. In some embodiments, the solvent is selected from tetrahydrofuran, N-methyl-2-pyrrolidone, methanol, isopropanol, and tert-butanol. In some embodiments, the solvent is isopropanol. In some embodiments, the solvent is tetrahydrofuran. In some embodiments, the solvent is N-methyl-2-pyrrolidone. In some embodiments, the solvent is methanol. In some embodiments, the solvent is tert-butanol.

In some embodiments of a process for the preparation of methyl (S)-2-((2-(2,6-difluoro-4-(methylcarbamoyl)phenyl)-7-methylimidazo[1,2-a]pyridin-3-yl)methyl)morpholine-4-carboxylate (Compound 1), the compound with the structure:

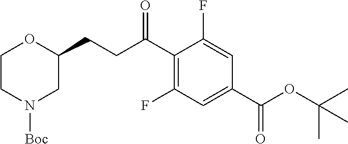 [SMILES: CC(C)(C)OC(=O)c1cc(F)c(C(=O)CC[C@H]2CN(C(=O)OC(C)(C)C)CCO2)c(F)c1]

is prepared by a process comprising contacting a compound with the structure:

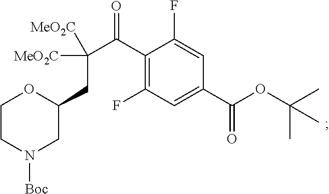 [SMILES: COC(=O)C(C[C@H]1CN(C(=O)OC(C)(C)C)CCO1)(C(=O)OC)C(=O)c1c(F)cc(C(=O)OC(C)(C)C)cc1F]

with lithium chloride and acetic acid.

In some embodiments of a process for the preparation of methyl (S)-2-((2-(2,6-difluoro-4-(methylcarbamoyl)phenyl)-7-methylimidazo[1,2-a]pyridin-3-yl)methyl)morpholine-4-carboxylate (Compound 1), the compound with the structure:

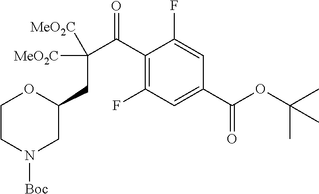 [SMILES: COC(=O)C(C[C@H]1CN(C(=O)OC(C)(C)C)CCO1)(C(=O)OC)C(=O)c1c(F)cc(C(=O)OC(C)(C)C)cc1F]

is prepared by a process comprising contacting a compound with the structure:

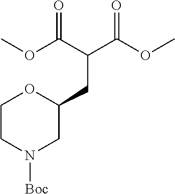 [SMILES: COC(=O)C(C[C@H]1CN(C(=O)OC(C)(C)C)CCO1)C(=O)OC]

with a compound with the structure:

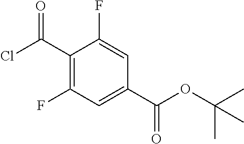 [SMILES: CC(C)(C)OC(=O)c1cc(F)c(C(=O)Cl)c(F)c1]

and a base, in the presence of a solvent. In some embodiments, the base is selected from sodium hydride, lithium bis(trimethylsilyl)amide, potassium tert-butoxide, and triethylamine/magnesium chloride. In some embodiments, the base is triethylamine/magnesium chloride. In some embodiments, the base is sodium hydride. In some embodiments, the base is lithium bis(trimethylsilyl)amide. In some embodiments, the base is potassium tert-butoxide. In some embodiments, the solvent is tetrahydrofuran or 2-methyltetrahydrofuran. In some embodiments, the solvent is 2-methyltetrahydrofuran. In some embodiments, the solvent is tetrahydrofuran.

In some embodiments of a process for the preparation of methyl (S)-2-((2-(2,6-difluoro-4-(methylcarbamoyl)phenyl)-7-methylimidazo[1,2-a]pyridin-3-yl)methyl)morpholine-4-carboxylate (Compound 1), the compound with the structure:

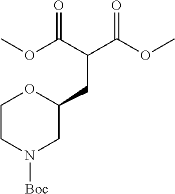 [SMILES: COC(=O)C(C[C@H]1CN(C(=O)OC(C)(C)C)CCO1)C(=O)OC]

is prepared by a process comprising contacting a compound with the structure:

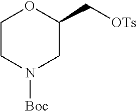 [SMILES: Cc1ccc(S(=O)(=O)OC[C@H]2CN(C(=O)OC(C)(C)C)CCO2)cc1]

with dimethylmalonate, potassium iodide, and a base. In some embodiments, the base is selected from sodium hydride, lithium bis(trimethylsilyl)amide, sodium bis(trimethylsilyl)amide, potassium bis(trimethylsilyl)amide, potassium carbonate, sodium carbonate, and cesium carbonate. In some embodiments, the base is cesium carbonate. In some embodiments, the base is sodium hydride. In some embodiments, the base is lithium bis(trimethylsilyl)amide. In some embodiments, the base is sodium bis(trimethylsilyl)amide. In some embodiments, the base is potassium bis(trimethylsilyl)amide. In some embodiments, the base is potassium carbonate. In some embodiments, the base is sodium carbonate.

In some embodiments of a process for the preparation of methyl (S)-2-((2-(2,6-difluoro-4-(methylcarbamoyl)phenyl)-7-methylimidazo[1,2-a]pyridin-3-yl)methyl)morpholine-4-carboxylate (Compound 1), the compound with the structure:

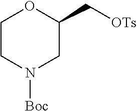 [SMILES: Cc1ccc(S(=O)(=O)OC[C@H]2CN(C(=O)OC(C)(C)C)CCO2)cc1]

is prepared by a process comprising contacting a compound with the structure:

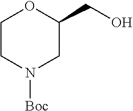 [SMILES: CC(C)(C)OC(=O)N1CCO[C@@H](CO)C1]

with toluenesulfonyl chloride and a base in the presence of a solvent. In some embodiments, the base is triethylamine. In some embodiments, the solvent is dichloromethane.

### Pharmaceutical Compositions and Methods of Administration

Administration of P2X3 antagonist as described herein can be in any pharmacological form including a therapeutically effective amount of an P2X3 antagonist alone or in combination with a pharmaceutically acceptable carrier.

Pharmaceutical compositions may be formulated in a conventional manner using one or more physiologically acceptable carriers including excipients and auxiliaries which facilitate processing of the active compounds into preparations which can be used pharmaceutically. Proper formulation is dependent upon the route of administration chosen. Additional details about suitable excipients for pharmaceutical compositions described herein may be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

A pharmaceutical composition, as used herein, refers to a mixture of Compound 1 described herein, with other chemical components, such as carriers, stabilizers, diluents, dispersing agents, suspending agents, thickening agents, and/or excipients. The pharmaceutical composition facilitates administration of the compound to an organism. In practicing the methods of treatment or use provided herein, therapeutically effective amounts of compounds described herein are administered in a pharmaceutical composition to a mammal having a disease, disorder, or condition to be treated. In some embodiments, the mammal is a human. A therapeutically effective amount can vary widely depending on the severity of the disease, the age and relative health of the subject, the potency of the compound used and other factors. Compound 1 can be used singly or in combination with one or more therapeutic agents as components of mixtures (as in combination therapy).

The pharmaceutical formulations described herein can be administered to a subject by multiple administration routes, including but not limited to, oral, parenteral (e.g., intravenous, subcutaneous, intramuscular), intranasal, buccal, topical, rectal, or transdermal administration routes. Moreover, the pharmaceutical compositions described herein, which include Compound 1 described herein, can be formulated into any suitable dosage form, including but not limited to, aqueous oral dispersions, liquids, gels, syrups, elixirs, slurries, suspensions, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations.

In some embodiments, Compound 1 is formulated in a tablet dosage form. In some embodiments, Compound 1 is formulated in a capsule dosage form. In some embodiments, Compound 1 is formulated in a suspension dosage form. In some embodiments, Compound 1 is formulated as powder-in-capsule dosage form. In some embodiments, Compound 1 is formulated as a powder-in-bottle for reconstitution as a suspension.

Pharmaceutical compositions including a compound described herein may be manufactured in a conventional manner, such as, by way of example only, by means of conventional mixing, dissolving, granulating, dragee-making, levigating, emulsifying, encapsulating, entrapping or compression processes.

Dose administration can be repeated depending upon the pharmacokinetic parameters of the dosage formulation and the route of administration used.

It is especially advantageous to formulate compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms are dictated by and directly dependent on (a) the unique characteristics of Compound 1 and the particular therapeutic effect to be achieved and (b) the limitations inherent in the art of compounding such an active compound for the treatment of sensitivity in individuals. The specific dose can be readily calculated by one of ordinary skill in the art, e.g., according to the approximate body weight or body surface area of the patient or the volume of body space to be occupied. The dose will also be calculated dependent upon the particular route of administration selected. Further refinement of the calculations necessary to determine the appropriate dosage for treatment is routinely made by those of ordinary skill in the art. Exact dosages are determined in conjunction with standard dose-response studies. It will be understood that the amount of the composition actually administered will be determined by a practitioner, in the light of the relevant circumstances including the condition or conditions to be treated, the choice of composition to be administered, the age, weight, and response of the individual patient, the severity of the patient's symptoms, and the chosen route of administration.

### Methods of Dosing and Treatment Regimens

The compounds described herein can be used in the preparation of medicaments for the modulation of P2X3, or for the treatment of diseases or conditions that would benefit, at least in part, from modulation of P2X3. In addition, a method for treating any of the diseases or conditions described herein in a subject in need of such treatment, involves administration of pharmaceutical compositions containing at least one compound described herein, or a pharmaceutically acceptable salt, or pharmaceutically acceptable solvate or hydrate thereof, in therapeutically effective amounts to said subject.

The compositions containing the compound(s) described herein can be administered for prophylactic and/or therapeutic treatments. In therapeutic applications, the compositions are administered to a patient already suffering from a disease or condition, in an amount sufficient to cure or at least partially arrest the symptoms of the disease or condition. Amounts effective for this use will depend on the severity and course of the disease or condition, previous therapy, the patient's health status, weight, and response to the drugs, and the judgment of the treating physician.

In prophylactic applications, compositions containing the compounds described herein are administered to a patient susceptible to or otherwise at risk of a particular disease, disorder or condition. Such an amount is defined to be a “prophylactically effective amount or dose.” In this use, the precise amounts also depend on the patient's state of health, weight, and the like. When used in a patient, effective amounts for this use will depend on the severity and course of the disease, disorder or condition, previous therapy, the patient's health status and response to the drugs, and the judgment of the treating physician.

In the case wherein the patient's condition does not improve, upon the doctor's discretion the administration of the compounds may be administered chronically, that is, for an extended period of time, including throughout the duration of the patient's life in order to ameliorate or otherwise control or limit the symptoms of the patient's disease or condition.

In the case wherein the patient's status does improve, upon the doctor's discretion the administration of the compounds may be given continuously; alternatively, the dose of drug being administered may be temporarily reduced or temporarily suspended for a certain length of time (i.e., a “drug holiday”). The length of the drug holiday can vary between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday may be from about 10% to about 100%, including, by way of example only, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

Once improvement of the patient's conditions has occurred, a maintenance dose is administered if necessary. Subsequently, the dosage or the frequency of administration, or both, can be reduced, as a function of the symptoms, to a level at which the improved disease, disorder or condition is retained. Patients can, however, require intermittent treatment on a long-term basis upon any recurrence of symptoms.

The amount of a given agent that will correspond to such an amount will vary depending upon factors such as the particular compound, disease or condition and its severity, the identity (e.g., weight) of the subject or host in need of treatment, but can nevertheless be determined in a manner recognized in the field according to the particular circumstances surrounding the case, including, e.g., the specific agent being administered, the route of administration, the condition being treated, and the subject or host being treated. In general, however, doses employed for adult human treatment will typically be in the range of about 0.01 mg per day to about 5000 mg per day, in some embodiments, about 1 mg per day to about 1500 mg per day. The desired dose may conveniently be presented in a single dose or as divided doses administered simultaneously (or over a short period of time) or at appropriate intervals, for example as two, three, four or more sub-doses per day.

The pharmaceutical composition described herein may be in unit dosage forms suitable for single administration of precise dosages. In unit dosage form, the formulation is divided into unit doses containing appropriate quantities of one or more compound. The unit dosage may be in the form of a package containing discrete quantities of the formulation. Non-limiting examples are packaged tablets or capsules, and powders in vials, capsules, bottles, or ampoules. Aqueous suspension compositions can be packaged in single-dose non-reclosable containers. Alternatively, multiple-dose reclosable containers can be used, in which case it is typical to include a preservative in the composition. By way of example only, formulations for parenteral injection may be presented in unit dosage form, which include, but are not limited to ampoules, or in multi-dose containers, with an added preservative.

### EXAMPLES

All chemicals, reagents, and solvents were purchased from commercial sources when available and used without further purification.

### Example 1: Synthesis of methyl (S)-2-((2-(2,6-difluoro-4-(methylcarbamoyl)phenyl)-7-methylimidazo[1,2-a]pyridin-3-yl)methyl)morpholine-4-carboxylate (Compound 1)

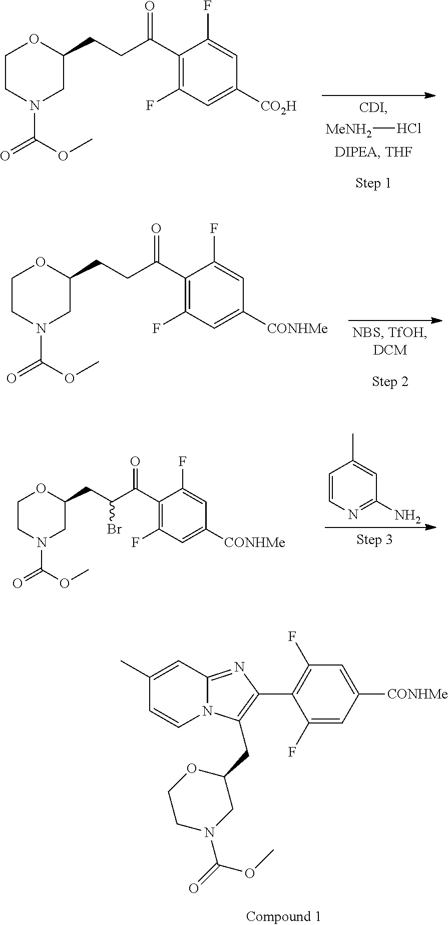

Step 1: To a mixture of (S)-3,5-difluoro-4-(3-(4-(methoxycarbonyl)morpholin-2-yl)propanoyl)benzoic acid in 5 V THF was added 2 eq. of CDI at 20-30° C. The reaction mixture was stirred for 0.5-1 h. then 2.5 eq. DIPEA was then added dropwise followed by 2.0 eq. MeNH₂·HCl in portions. The reaction mixture was stirred at that temperature for 2-4 h, then quenched by adding dropwise 2.5 V H₂O at 20-30° C. The resultant mixture was extracted twice with DCM. The combined organic layers were washed twice with 2.5× 20% NH₄Cl solution and twice with water. The solvent of the organic layer was then swapped to EtOAc. The mixture was then stirred at 45-55° C. for 1-2 h, then at 20-30° C. for 1-2h. The resulting suspension was further cooled to 0-10° C. and stirred at that temperature for 1-2 h. The mixture was filtered, and the wet cake was slurried in EtOAc. The mixture was then filtered and the wet cake was dried under vacuum at 35-55° C. to afford intermediate methyl (S)-2-(3-(2,6-difluoro-4-(methylcarbamoyl)phenyl)-3-oxopropyl)morpholine-4-carboxylate. LCMS (ESI, m/z): 371.1 [M+H]⁺. ¹H NMR (400 MHz, CDCl₃) δ 1.77-1.99 (m, 2H), 2.67 (br s, 1H), 2.91-3.10 (m, 6H), 3.36-3.52 (m, 2H), 3.72 (s, 3H), 3.79-4.05 (m, 3H), 6.25 (br s, 1H), 7.31-7.40 (m, 2H).

Step 2: To a solution of methyl (S)-2-(3-(2,6-difluoro-4-(methylcarbamoyl)phenyl)-3-oxopropyl)morpholine-4-carboxylate in DCM was added NBS (1.0 eq). The reaction mixture was cooled to −5 to 5° C. then trifluoromethanesulfonic acid (1.0 eq.) was added dropwise. The mixture was stirred at 25-30° C. and upon reaction completion, a 7% NaHCO₃ aq. solution was charged at −5 to 5° C. and then ascorbic acid was added to adjust pH=5-6. The layers were separated then the aqueous layer was back extracted twice with DCM. The combined organic layers were washed with water and the resulting DCM solution of methyl (2S)-2-(2-bromo-3-(2,6-difluoro-4-(methylcarbamoyl)phenyl)-3-oxopropyl)morpholine-4-carboxylate was used directly into the next step. LCMS (ESI, m/z): 449.2 and 451.1 [M+H]⁺. ¹H NMR (400 MHz, CDCl₃) δ 1.57 (br s, 1H), 2.04-2.24 (m, 1H), 2.25-2.40 (m, 1H), 2.56 (br dd, J=13.51, 10.38 Hz, 1H), 2.77 (br d, J=3.75 Hz, 1H), 2.88-3.11 (m, 4H), 3.35-3.59 (m, 2H), 3.61-3.78 (m, 4H), 3.80-4.18 (m, 3H), 5.18 (td, J=10.19, 3.50 Hz, 1H), 6.08-6.65 (m, 1H), 7.38 (br t, J=7.38 Hz, 2H).

Step 3: A solution of methyl (2S)-2-(2-bromo-3-(2,6-difluoro-4-(methylcarbamoyl)phenyl)-3-oxopropyl)morpholine-4-carboxylate in DCM was concentrated to dryness and the solvent was switched to ACN. To the resulting solution was added 4-methylpyridin-2-amine (10.0 eq), then the reaction mixture was heated to 30° C. and stirred for 24 h at this temperature. The reaction mixture was then heated to 50° C. for 64h and upon completion, the reaction mixture was concentrated, and the solvent switched to DCM. Water was added, then the mixture was cooled to 0-10° C. The pH was adjusted to 4.0-4.5 with H₂SO₄ then the layers were separated. DCM was added to the aqueous layer and the pH was adjusted to 3.0-3.5 with H₂SO₄ at 0-10° C. The layers were separated. DCM was added to the aqueous layer and the pH was adjusted to 2.0-2.5 with H₂SO₄ at 0-10° C. The layers were separated. The combined organic layers were concentrated, and the solvent switched to THF. To the resulting THF solution was charged 10% NaOH aq. solution (1.5 eq) and NaBH₄ (0.45 eq). The mixture was stirred at 20° C. for 2-5h. The reaction mixture was quenched with water at 0-10° C. and extracted with MTBE then DCM. To the aqueous layer was added DCM, and the pH was adjusted to 5.6-5.8 with 2 N HCl at 0-10° C. The layers were separated, and the aqueous layer was extracted with DCM three times. The combined organic layers were concentrated, and the solvent switched to MeOH. The resulting mixture was stirred at 55-65° C. for 0.5h, then cooled to −5-5° C. and stirred at that temperature for 2h. Water (10V) was added dropwise and the mixture was stirred for 3 h at this temperature. The suspension was filtered and the wet cake washed with water then dissolved in MeOH (7V). The solution was heated to 55-65° C. for 1 h, then cooled to 20-30° C. The mixture was partially concentrated then cooled to −5-5° C. Water was added drop wise at −5-5° C. and the mixture was stirred for 1 h at this temperature. The suspension was filtered and the resulting wet cake was washed with water then dried under vacuum at 55-65° C. to afford methyl (S)-2-((2-(2,6-difluoro-4-(methylcarbamoyl)phenyl)-7-methylimidazo[1,2-a]pyridin-3-yl)methyl)morpholine-4-carboxylate (Compound 1) as a white solid. LCMS (ESI, m/z): 459.3 [M+H]⁺. ¹H NMR (400 MHz, DMSO-d6) δ ppm 2.38 (s, 3H), 2.82 (d, J=4.27 Hz, 4H), 2.98-3.09 (m, 2H), 3.22 (td, J=11.67, 2.76 Hz, 1H), 3.47 (br d, J=3.51 Hz, 1H), 3.55 (s, 3H), 3.62 (br d, J=9.29 Hz, 2H), 3.73 (br d, J=12.30 Hz, 1H), 6.84 (dd, J=7.03, 1.51 Hz, 1H), 7.35 (s, 1H), 7.65 (d, J=8.03 Hz, 2H), 8.42 (d, J=7.03 Hz, 1H), 8.69 (br d, J=4.52 Hz, 1H).

### Example 2: Potency and Selectivity for Human P2X3 and P2X2/3 Receptors

The ability of Compound 1 described herein to act as an antagonist of the P2X3 and P2X2/P2X3 channel (encoded by the human P2RX2 and P2RX3 genes, stably expressed in HEK293 cells) was evaluated with a Fluo-8 calcium kit. Compound 1 was evaluated at twelve concentrations.

For the antagonist effect assessment, the cells were pre-incubated with Compound 1 for 20 minutes, then stimulated with the P2X3 and P2X2/P2X3 agonist α,β-methyleneATP (meATP) at final concentrations of 3 μM and 30 μM. Four minutes fifty seconds after addition of meATP, ionomycin was added at a final concentration of 5 μM in order to obtain the maximum calcium influx and fluorescence signal possible from the cells. Fluorescence was recorded continuously for 10 minutes, starting 10 seconds prior to the addition of meATP. IC₅₀s obtained using the above methods indicate that Compound 1 is a selective P2X3 antagonist (P2X3 IC₅₀=11 nM; P2X2/3 IC₅₀>30 μM).

The examples and embodiments described herein are for illustrative purposes only and in some embodiments, various modifications or changes are to be included within the purview of disclosure and scope of the appended claims.

1-2. (canceled)

3. A process for the preparation of methyl (S)-2-((2-(2,6-difluoro-4-(methylcarbamoyl)phenyl)-7-methylimidazo[1,2-a]pyridin-3-yl)methyl)morpholine-4-carboxylate (Compound 1): 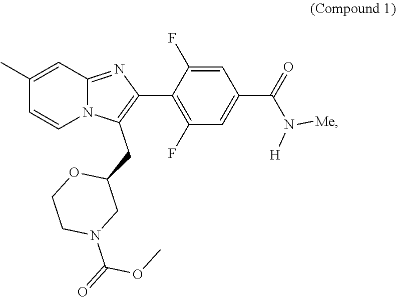 [SMILES: CNC(=O)c1cc(F)c(-c2nc3cc(C)ccn3c2C[C@H]2CN(C(=O)OC)CCO2)c(F)c1]
comprising contacting a compound with the structure: 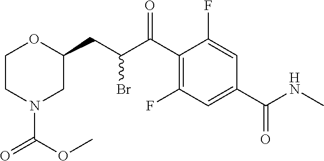 [SMILES: CNC(=O)c1cc(F)c(C(=O)C(Br)C[C@H]2CN(C(=O)OC)CCO2)c(F)c1]
with 4-methylpyridin-2-amine in the presence of a solvent, optionally wherein the solvent is acetonitrile or acetonitrile containing water.

4. (canceled)

5. The process of claim 3, wherein the compound with the structure: 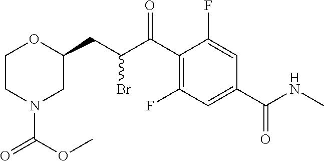 [SMILES: CNC(=O)c1cc(F)c(C(=O)C(Br)C[C@H]2CN(C(=O)OC)CCO2)c(F)c1]
is prepared by a process comprising contacting a compound with the structure: 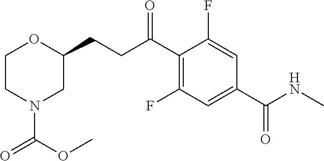 [SMILES: CNC(=O)c1cc(F)c(C(=O)CC[C@H]2CN(C(=O)OC)CCO2)c(F)c1]
with a brominating reagent.

6. The process of claim 5, wherein the brominating agent is N-bromosuccinimide in the presence of acid, optionally wherein the acid is trifluoromethanesulfonic acid.

7. (canceled)

8. The process of claim 5, wherein the compound with the structure: 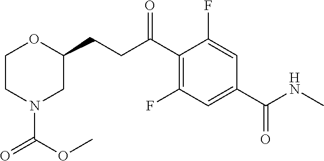 [SMILES: CNC(=O)c1cc(F)c(C(=O)CC[C@H]2CN(C(=O)OC)CCO2)c(F)c1]
is prepared by a process comprising contacting a compound with the structure: 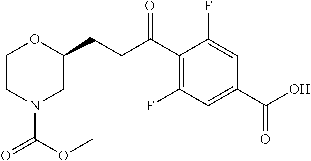 [SMILES: COC(=O)N1CCO[C@@H](CCC(=O)c2c(F)cc(C(=O)O)cc2F)C1]
with an amide coupling reagent and methylamine or a salt of methylamine.

9-10. (canceled)

11. The process of claim 3, wherein the compound with the structure: 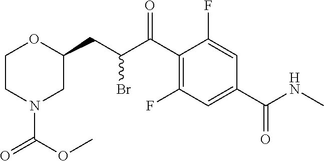 [SMILES: CNC(=O)c1cc(F)c(C(=O)C(Br)C[C@H]2CN(C(=O)OC)CCO2)c(F)c1]
is prepared by a process comprising contacting a compound with the structure: 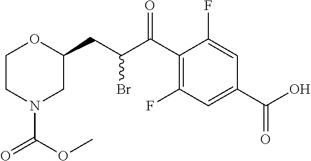 [SMILES: COC(=O)N1CCO[C@@H](CC(Br)C(=O)c2c(F)cc(C(=O)O)cc2F)C1]
with an amide coupling reagent and methylamine or a salt of methylamine.

12. The process of claim 11, wherein the amide coupling reagent is carbonyldiimidazole or propanephosphonic acid anhydride (T3P).

13. The process of claim 8, wherein the amide coupling reagent is carbonyldiimidazole or propanephosphonic acid anhydride (T3P).

14. The process of claim 11, wherein the compound with the structure: 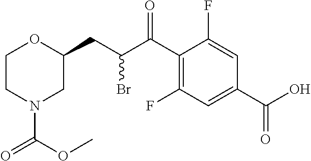 [SMILES: COC(=O)N1CCO[C@@H](CC(Br)C(=O)c2c(F)cc(C(=O)O)cc2F)C1]
is prepared by a process comprising contacting a compound with the structure: 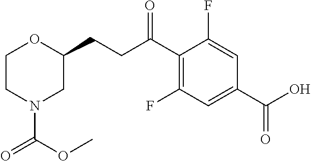 [SMILES: COC(=O)N1CCO[C@@H](CCC(=O)c2c(F)cc(C(=O)O)cc2F)C1]
with a brominating agent.

15. The process of claim 14, wherein the brominating agent is N-bromosuccinimide in the presence of acid, optionally wherein the acid is trifluoromethanesulfonic acid.

16. (canceled)

17. The process of claim 14, wherein the compound with the structure: 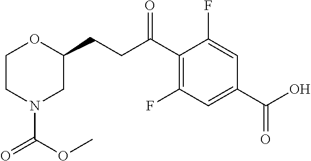 [SMILES: COC(=O)N1CCO[C@@H](CCC(=O)c2c(F)cc(C(=O)O)cc2F)C1]
is prepared by a process comprising contacting a compound with the structure: 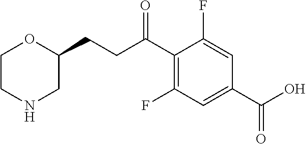 [SMILES: O=C(O)c1cc(F)c(C(=O)CC[C@H]2CNCCO2)c(F)c1]
with methyl chloroformate and a base, optionally wherein the base is aqueous sodium bicarbonate.

18. (canceled)

19. The process of claim 17, wherein the compound with the structure: 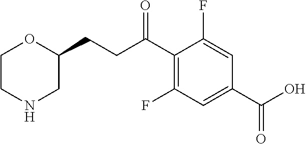 [SMILES: O=C(O)c1cc(F)c(C(=O)CC[C@H]2CNCCO2)c(F)c1]
is prepared by a process comprising contacting a compound with the structure: 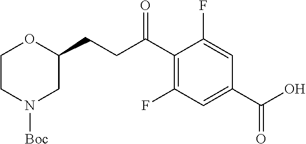 [SMILES: CC(C)(C)OC(=O)N1CCO[C@@H](CCC(=O)c2c(F)cc(C(=O)O)cc2F)C1]
with a deprotection reagent selected from 1) hydrogen chloride in the presence of a solvent, 2) trifluoroacetic acid, and 3) phosphoric acid.

20. (canceled)

21. The process of claim 19, wherein the compound with the structure: 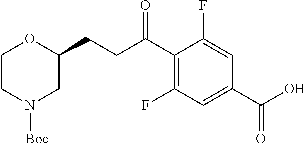 [SMILES: CC(C)(C)OC(=O)N1CCO[C@@H](CCC(=O)c2c(F)cc(C(=O)O)cc2F)C1]
is prepared by a process comprising contacting a compound with the structure: 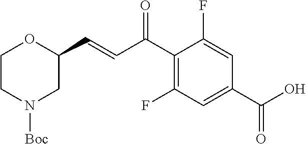 [SMILES: CC(C)(C)OC(=O)N1CCO[C@@H](/C=C/C(=O)c2c(F)cc(C(=O)O)cc2F)C1]
with a hydrogenation catalyst and hydrogen.

22-23. (canceled)

24. The process of claim 21, wherein the compound with the structure: 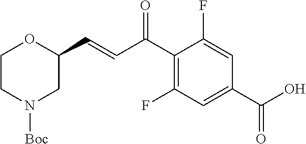 [SMILES: CC(C)(C)OC(=O)N1CCO[C@@H](/C=C/C(=O)c2c(F)cc(C(=O)O)cc2F)C1]
is prepared by a process comprising contacting a compound with the structure: 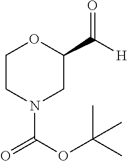
with a compound with the structure: 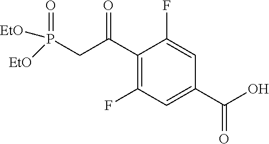 [SMILES: CCOP(=O)(CC(=O)c1c(F)cc(C(=O)O)cc1F)OCC]
with a base, wherein the base is optionally a mixture of aqueous potassium bicarbonate and potassium carbonate or the base is potassium phosphate.

25. (canceled)

26. The process of claim 19, wherein the compound with the structure: 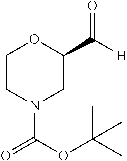
is prepared by a process comprising contacting a compound with the structure: 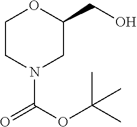
with an oxidizing agent, wherein the oxidizing agent is optionally selected from 2,2,6,6-tetramethylpiperidine 1-oxyl, T3P, and trichloroisocyanuric acid (TCCA).

27. (canceled)

28. The process of claim 19, wherein the compound with the structure: 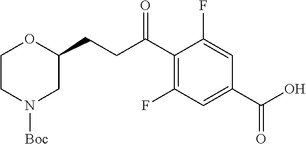 [SMILES: CC(C)(C)OC(=O)N1CCO[C@@H](CCC(=O)c2c(F)cc(C(=O)O)cc2F)C1]
is prepared by a process comprising contacting a compound with the structure: 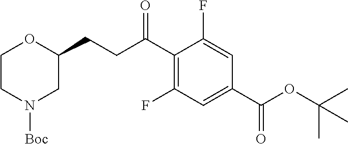 [SMILES: CC(C)(C)OC(=O)c1cc(F)c(C(=O)CC[C@H]2CN(C(=O)OC(C)(C)C)CCO2)c(F)c1]
with a base in a solvent.

29-31. (canceled)

32. The process of claim 28, wherein the compound with the structure: 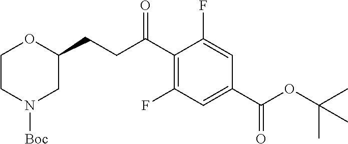 [SMILES: CC(C)(C)OC(=O)c1cc(F)c(C(=O)CC[C@H]2CN(C(=O)OC(C)(C)C)CCO2)c(F)c1]
is prepared by a process comprising contacting a compound with the structure: 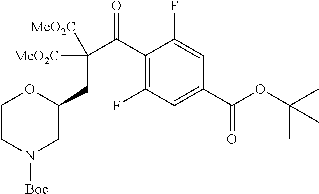 [SMILES: COC(=O)C(C[C@H]1CN(C(=O)OC(C)(C)C)CCO1)(C(=O)OC)C(=O)c1c(F)cc(C(=O)OC(C)(C)C)cc1F]
with lithium chloride and acetic acid.

33. The process of claim 32, wherein the compound with the structure: 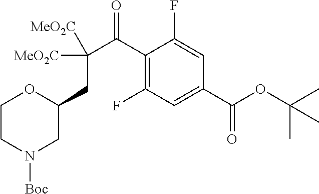 [SMILES: COC(=O)C(C[C@H]1CN(C(=O)OC(C)(C)C)CCO1)(C(=O)OC)C(=O)c1c(F)cc(C(=O)OC(C)(C)C)cc1F]
is prepared by a process comprising contacting a compound with the structure: 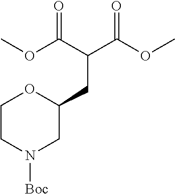 [SMILES: COC(=O)C(C[C@H]1CN(C(=O)OC(C)(C)C)CCO1)C(=O)OC]
with a compound with the structure: 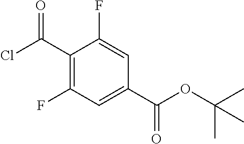 [SMILES: CC(C)(C)OC(=O)c1cc(F)c(C(=O)Cl)c(F)c1]
and a base, in the presence of a solvent.

34. The process of claim 33, wherein the base is selected from sodium hydride, lithium bis(trimethylsilyl)amide, potassium tert-butoxide, and triethylamine/magnesium chloride.

35-37. (canceled)

38. The process of claim 33, wherein the compound with the structure: 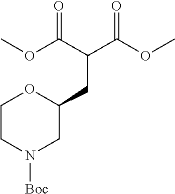 [SMILES: COC(=O)C(C[C@H]1CN(C(=O)OC(C)(C)C)CCO1)C(=O)OC]
is prepared by a process comprising contacting a compound with the structure: 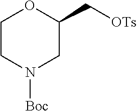 [SMILES: Cc1ccc(S(=O)(=O)OC[C@H]2CN(C(=O)OC(C)(C)C)CCO2)cc1]
with dimethylmalonate, potassium iodide, and a base.

39. The process of claim 38, wherein the base is selected from sodium hydride, lithium bis(trimethylsilyl)amide, sodium bis(trimethylsilyl)amide, potassium bis(trimethylsilyl)amide, potassium carbonate, sodium carbonate, and cesium carbonate.

40-45. (canceled)

46. A compound having the structure: 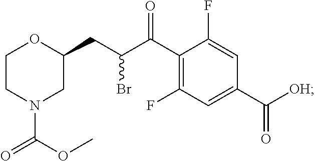 [SMILES: COC(=O)N1CCO[C@@H](CC(Br)C(=O)c2c(F)cc(C(=O)O)cc2F)C1]
or a pharmaceutically acceptable salt thereof.
